(19) United States
(12) Patent Application Publication
(10) Pub. No.: US 2026/0234480 A1
(43) Pub. Date: Aug. 13, 2026

(54) METHOD FOR OBTAINING A WAX FROM A PYROLYSIS RESIDUE

(71) Applicant: OMV DOWNSTREAM GMBH, Wien (AT)

(72) Inventors: Andreas LECHLEITNER, Wien (AT); Matthias MASTALIR, Wien (AT); Alexander WEILER, Wien (AT)

(21) Appl. No.: 19/475,009

(22) PCT Filed: Nov. 7, 2023
(86) PCT No.: PCT/EP2023/080918

§ 371 Date: Oct. 13, 2025

(30) Foreign Application Priority Data
Apr. 18, 2023 (EP) 23168526.4

(51) Int. Cl. C10G 21/14; C07C 69/26; C08L 91/06; C10G 1/00; C10G 1/02; C10G 31/09; C11B 11/00

(52) U.S. Cl. CPC C10G 21/14; C07C 69/26; C08L 91/06; C10G 1/002; C10G 1/02; C10G 31/09; C11B 11/00; C10G 2300/1003

(57) ABSTRACT

A method for obtaining a wax from a pyrolysis residue includes providing the pyrolysis residue, where the pyrolysis residue includes the wax and a solid material, and where the solid material includes an inorganic salt, a ceramic raw material, an asphaltene, a tar and/or a coke. A solvent is added to the pyrolysis residue to obtain a high-solid mixture with the solid material and the solvent, where the wax is at least partially dissolved in the solvent, and at least a portion of the solid material is separated from the high-solid mixture to obtain a low-solid mixture. The method also includes separating at least a portion of the wax from the low-solid mixture. A device is described for obtaining a wax from a pyrolysis residue using the method.

The invention relates to a method and a device for obtaining a wax from a pyrolysis residue.

Methods for pyrolyzing a starting material, in particular a plastic, are known in the prior art. The focus is regularly on the recovery of products from a gaseous fraction of the pyrolysis product obtained. The pyrolysis residue is produced as a waste product and is normally used for energy purposes.

Such a pyrolysis method is disclosed, for example, in JPH 09125073 A. In this case, a waste plastic is pyrolyzed in a pyrolysis reactor in order to obtain a pyrolysis product comprising a gaseous fraction and a pyrolysis residue. The gaseous fraction is cooled and condensed into a product oil. Various fractionated oils can be obtained from this product oil by distillation. A solvent, which can be a product oil or a fractionated oil, is added to the pyrolysis residue. Alternatively, kerosene, light oil, heavy oil, benzene, toluene or xylene may be used as solvents. A portion of the pyrolysis residue should dissolve in the solvent, which should enable a solid, coke-containing fraction contained in the pyrolysis residue to be separated. The solvent with the portion of the pyrolysis residue dissolved therein is then recycled to the pyrolysis reactor for renewed pyrolysis.

WO 2023/285472 A2 describes a method for the pyrolysis of a plastic waste, in which a resulting pyrolysis gas is separated from solid materials (e.g. coke). The pyrolysis gas is then condensed to obtain a pyrolysis oil fraction, from which a wax is then separated.

WO 2017/168165 A1 relates to a method for producing a lubricant by pyrolysis of a plastic. WO 2021/115982 A1 describes a method for obtaining aliphatic hydrocarbons from a liquid hydrocarbon stream. US 2021/0332300 A1 is directed to a method for converting plastic waste into recycled products for use in the polymerization of propylene. WO 2009/086107 A2 provides a method for converting heavy oil into lower-boiling hydrocarbon products. The subject matter of US 2009/0057192 A1 is a method for generating an improved input stream for a deasphalting process. U.S. Pat. No. 3,720,599 A discloses a method for dewaxing petroleum.

Despite the methods known from the prior art, the utilization of pyrolysis residues, in particular from plastic pyrolysis, has not yet been satisfactorily solved. There is therefore a need for alternative, better methods for recycling such pyrolysis residues. It is an object of the invention to provide such a method.

The present invention relates to a method for obtaining a wax from a pyrolysis residue, comprising the steps of
(a) providing the pyrolysis residue, wherein the pyrolysis residue comprises the wax and a solid material, preferably wherein the solid material comprises an inorganic salt, a ceramic raw material, an asphaltene, a tar and/or a coke,
(b) adding a solvent to the pyrolysis residue to obtain a high-solid mixture comprising the solid material and the solvent, wherein the wax is at least partially dissolved in the solvent,
(c) separating at least a portion of the solid material from the high-solid mixture to obtain a low-solid mixture, and
(d) separating at least a portion of the wax from the low-solid mixture.

The invention is based on the finding that a pyrolysis residue, in particular a pyrolysis residue obtained by plastic pyrolysis, is a valuable source of a wax. By means of the method according to the invention, not only can at least a portion of the wax contained in the pyrolysis residue be obtained, but also at least a portion of the solid material contained in the pyrolysis residue. The method can therefore relieve the environment, as greenhouse gas emissions can be reduced and the amount of waste can be reduced. Overall, this can significantly reduce re-source consumption and strengthen the circular economy, which can also bring economic benefits. In particular, the wax obtained can be a sustainable alternative to wax obtained directly from petroleum.

The pyrolysis residue can be obtained by pyrolyzing a plastic, in particular a waste plastic. The pyrolysis can take place in a pyrolysis reactor, preferably at a temperature of 300 to 500° C., in particular 350 to 450° C. As a result, a good balance between cost-effectiveness and method efficiency can be achieved.

The pyrolysis may be thermal pyrolysis (i.e., thermal cracking, without the addition of a catalyst) and/or catalytic pyrolysis (i.e., catalytic cracking). Thermal pyrolysis is preferred to avoid any contamination of the wax and/or solid material due to catalyst components.

The pyrolysis can be carried out largely in the absence of oxygen, in particular in an inert atmosphere, for example under nitrogen. Due to a lack of oxygen or exclusion of oxygen, complete combustion can be prevented and a polymer contained in the plastic can be cleaved or depolymerized.

The plastic preferably comprises a polyolefin and/or a polystyrene (PS), wherein the polyolefin may comprise a polyethylene (PE) and/or a polypropylene (PP). The plastic preferably comprises the polyolefin and/or the polystyrene in an amount of at least 65% by weight, more preferably at least 70% by weight, in particular at least 90% by weight, based on the total weight of the plastic. As a result, a pyrolysis residue can be obtained which comprises a significant proportion of an aliphatic hydrocarbon (or a mixture of a plurality of aliphatic hydrocarbons), i.e. a significant proportion of the wax.

The plastic preferably comprises at least 20% by weight of the polyolefin, more preferably at least 50% by weight, even more preferably at least 70% by weight, in particular at least 90% by weight, based on the total weight of the plastic. As the proportion of polyolefin increases, the amount of wax obtained by the method according to the invention can be increased, which can improve the economics of the method. The plastic may comprise a further polymer from the group consisting of thermoplastics, duromers and/or elastomers, in particular an acrylonitrile-butadiene-styrene copolymer (ABS), a polyvinyl chloride (PVC), a polyamide (PA) and/or a polyester.

Before pyrolysis, the plastic can be plasticized, for example in a mixer, in particular in an extruder. The plastic is preferably heated to a temperature of at least 120° C. in order to plasticize it, more preferably to a temperature of 200 to 500° C., even more preferably of 400 to 470° C. Then the subsequent pyrolysis can be carried out more energy-efficiently and in a shorter time. In the extruder, the plastic can also be degassed to produce a uniform mass without gas inclusions, so that a homogeneous pyrolysis product can be obtained by the subsequent pyrolysis.

Before the pyrolysis, a diluent for viscosity reduction can be added to the plastic, in particular the plasticized plastic. The diluent is preferably added to the plastic in an amount of at least 5% by weight, more preferably at least 9% by weight, based on the total weight of the plastic. The ratio of plastic to diluent is preferably at least 1:4, preferably at least 1:9. By adding the diluent to the plastic, the mobility of polymer chains can be increased at a given temperature, so that heat input into the plastic can be improved during pyrolysis. Furthermore, due to the viscosity reduction, the risk of plastic over-heating in wall areas of the pyrolysis reactor can be reduced, since it is usually heated by a heating device arranged near an outer wall of the pyrolysis reactor. The risk of coking of the plastic during pyrolysis can also be reduced by reducing the viscosity.

By adding the diluent to the plastic, its viscosity can preferably be reduced by at least 30%, more preferably by at least 50%, particularly preferably by at least 80%, based on the viscosity of the plastic without diluent under the same measurement conditions, in particular at a temperature in the range from 180 to 240° C. This can improve the pumpability of the plastic, which can facilitate its processing.

When the diluent is added, the plastic preferably has a temperature of at least 120° C., more preferably a temperature of 150 to 300° C., in particular 200 to 300° C. Alternatively or additionally, the diluent can be heated to a temperature of preferably at least 120° C., more preferably at least 150° C., in particular to a temperature of 200 to 300° C., before being added to the plastic. By increasing the temperature of the plastic and/or the diluent, the diluent can be mixed into the plastic more quickly and efficiently. The subsequent pyrolysis can also be carried out more energy-efficiently and faster.

The diluent can be added to the plastic by means of a feed device. The feed device may have a metering device, such as a metering pump. For example, the plastic, in particular the plasticized plastic, can be fed to a mixer, e.g. a static mixer, and mixed there with the diluent. If the plastic is plasticized in an extruder, the diluent can be added directly in the extruder. For this purpose, the feed device can be arranged, for example, in the compression zone or mixing zone of the extruder.

The diluent may comprise a hydrocarbon selected from an alkane, a cycloalkane, and/or an aromatic. As a result of the pyrolysis, such a diluent can be converted into a gaseous and/or liquid product, which can be at least partially separated from the pyrolysis residue and reused. The diluent preferably comprises at least a portion of a liquid fraction of the pyrolysis residue. This can be separated, for example, in a hydrocyclone. Alternatively or additionally, the diluent may comprise a fraction obtained from crude oil, in particular a heavy oil. The heavy oil can be an oil obtained from petroleum in a petroleum refinery, e.g. a residual oil from a pyrolysis plant. The heavy oil preferably has a proportion of an aromatic hydrocarbon of at least 25% by weight, based on the total weight of the heavy oil.

The diluent preferably has a boiling temperature (or a lower end of a boiling range) of at least 300° C., in particular at least 350° C. As a result, the diluent can be prevented from evaporating immediately after a mixture of plastic and diluent has been introduced into the pyrolysis reactor, but evaporation, cleavage and/or depolymerization of the diluent can only take place with the progressive residence time of the mixture in the pyrolysis reactor and the associated heating of the mixture. As a result, a homogeneous pyrolysis product can be obtained.

Within the scope of the invention, it has surprisingly been found that the addition of a diluent during pyrolysis leads to a particularly wax-rich pyrolysis residue. This is particularly advantageous in connection with the method according to the invention, since a larger amount of wax can still be obtained in this way.

Apart from the wax and the solid material, the pyrolysis residue provided in step (a) of the method may also comprise a liquid fraction. The proportion of the liquid fraction is preferably at most 95% by weight, more preferably 30 to 95% by weight, in particular 50 to 88% by weight, based on the total weight of the pyrolysis residue. As the proportion of the liquid fraction decreases, at least a portion of the wax can dissolve more quickly in the solvent, or a smaller amount of solvent must be added to the pyrolysis residue in order to dissolve the wax at least partially in the solvent. As a result, the separation of at least a portion of the wax from the low-solid mixture can also be facilitated. Thus, the method can be designed more efficiently.

The pyrolysis residue provided in step (a) of the method may be obtained by pyrolyzing the plastic in a pyrolysis reactor and subsequently increasing a solids concentration of the pyrolysis residue. This can be advantageous in order to reduce the proportion of the liquid fraction of the pyrolysis residue obtained to preferably a maximum +95% by weight, more preferably to 30 to 95% by weight, in particular to 50 to 88% by weight, based on the total weight of the pyrolysis residue. In order to increase the solids concentration, a separation device, preferably a hydrocyclone, can be used, which can be connected downstream of the pyrolysis reactor.

If the pyrolysis residue is obtained by pyrolysis of a plastic, a gaseous fraction can be separated from the pyrolysis residue after the pyrolysis and before step (a) of the method. The separation of the gaseous fraction can take place by means of evaporation, for example in a hydrocyclone, which can be connected downstream of the pyrolysis reactor.

The separation of the gaseous fraction from the pyrolysis residue and the increase in the solids concentration of the pyrolysis residue can be carried out, for example, with a separation device or with a plurality of separation devices connected in series. Advantageously, both the separation of the gaseous fraction from the pyrolysis residue and the increase in the solids concentration of the pyrolysis residue take place in only one method step by means of a hydrocyclone, which may be connected downstream of the pyrolysis reactor. Such a hydrocyclone is described in the application WO 2023/036751A1. By introducing a mixture comprising the pyrolysis residue and the gaseous fraction via an inlet arranged in an upper region of a jacket of the hydrocyclone, a vortex flow can be generated in the hydrocyclone, as a result of which the gaseous fraction can be separated from the pyrolysis residue and discharged via an outlet arranged in the upper region of the hydrocyclone (e.g. on its ceiling). The pyrolysis residue can then be discharged in the direction of a bottom of the hydrocyclone due to gravity, wherein a tangential velocity of a vortex flow that forms can continuously increase. As a result, at least a portion of the pyrolysis residue, in particular at least a portion of the pyrolysis residue at least partially comprising the wax and the solid material, can be discharged via an outlet arranged in the bottom of the hydrocyclone, while at least a portion of the liquid fraction of the pyrolysis residue passes into an inner container arranged in the hydrocyclone and can be discharged from there via an outlet, e.g. for further use as a diluent. The pyrolysis residue discharged via the outlet disposed in the bottom of the hydrocyclone can then be provided according to step (a) of the method. The hydrocyclone is preferably operated at a temperature in the range from 300 to 450° C., more preferably from 320 to 420° C., particularly preferably from 360 to 400° C.

The pyrolysis residue may be cooled prior to addition of the solvent, either prior to step (a) of the method, or between steps (a) and (b). Cooling can be carried out by means of a cooling unit. The pyrolysis residue is preferably cooled to a temperature of not more than 240° C., more preferably not more than 220° C., particularly preferably not more than 180° C. The cooling of the pyrolysis residue can be particularly advantageous if the pyrolysis residue is obtained by immediately preceding processing of a plastic (by pyrolysis and, if necessary, subsequent separation of a gaseous fraction from the pyrolysis residue and/or increasing the solids concentration of the pyrolysis residue). By cooling the pyrolysis residue, undesired evaporation and/or decomposition of the solvent during addition to the pyrolysis residue can be reduced or completely prevented. The pyrolysis residue is preferably cooled to a temperature of not less than 80° C., more preferably not less than 100° C., particularly preferably not less than 120° C. If this temperature is not undershot, this has the advantage that the solubility of the wax in the solvent is better.

The solvent may be added to the pyrolysis residue in step (b) of the method by means of an introduction device. The introduction device may have a metering device, such as a metering pump. To add the solvent, the pyrolysis residue can be fed to a container to which the introduction device can be connected. The container can be heatable so that the dissolving of the wax in the solvent can take place at a certain temperature.

The wax is preferably dissolved in the solvent at a temperature of at least 80° C., more preferably at least 100° C., even more preferably at least 120° C., even more preferably 140° C., in particular at least 160° C. The wax is preferably dissolved at a temperature in the range from 80 to 240° C., more preferably from 100 to 160° C., in particular 100 to 140° C. This ensures good method efficiency, since on the one hand the wax can dissolve well and quickly in the solvent, and on the other hand the method can be carried out quickly and energy-efficiently. The wax can dissolve particularly well in the solvent even at a temperature of 100 to 120° C., while a temperature of 120 to 140° C. can be optimal in terms of procedure.

Dissolution of the wax in the solvent can preferably take place at a pressure in the range from 1 to 25 bar, in particular in the range from 5 to 16 bar. As a result, the method can be further improved and rapid dissolution of the wax in the solvent can be achieved.

The solvent can preferably have a boiling temperature (or boiling range) in the range from 20 to 270° C., more preferably from 20 to 250° C., even more preferably from 35 to 255° C., even more preferably from 20 to 200° C., in particular from 50 to 150° C. As a result, the method can be carried out efficiently. In particular, the solvent preferably has a boiling temperature (or an upper end of a boiling range) that is at least 10° C., more preferably at least 30° C., in particular at least 50° C., below the boiling temperature of the wax (or below a lower end of the boiling range of the wax). As a result, the wax can be easily separated from the low-solid mixture. The boiling temperature (or boiling range) of the solvent can be determined with the standard ASTM D 5399-09: 2017 or ASTM D 2887-22: 2022.

The solvent preferably comprises at least 10% by weight, more preferably at least 20% by weight, even more preferably at least 50% by weight, even more preferably at least 70% by weight, in particular at least 90% by weight, of an aliphatic hydrocarbon, based on the total weight of the solvent. The solvent preferably consists of an aliphatic hydrocarbon, or of a mixture consisting of two or more aliphatic hydrocarbons. As the proportion of aliphatic hydrocarbon increases, not only can the solvent's ability to dissolve the wax improve, but the solubility of the solid material in the solvent can also be reduced. In the case of a high proportion of an aromatic in the solvent, on the other hand, the solid material, in particular an asphaltene or a tar, can dissolve well in the solvent, which can make the subsequent separation of the wax more difficult, or as a result of which the separated wax can be contaminated. With a high proportion of a cycloalkane in the solvent, the wax can dissolve well, but the yield can be comparatively low.

The aliphatic hydrocarbon is preferably selected from the group of aliphatic hydrocarbons having up to 15 carbon atoms per molecule, in particular from 4 to 12 carbon atoms per molecule. As a result, the method can be carried out efficiently, and good separation of the wax from the low-solid mixture can be made possible.

The aliphatic hydrocarbon is preferably selected from n-pentane, n-hexane, cyclo-hexane, n-heptane, iso-dodecane, tetradecane, an isomer thereof, or a mixture thereof. Since the boiling point of these aliphatic hydrocarbons is in the range from 35 to 255° C., the method can be carried out efficiently and economically. Furthermore, the wax can dissolve well in these solvents due to the low molecular weight of these solvents. Preferably, the solvent comprises 10 to 80% by weight of an aliphatic hydrocarbon having up to 15 carbon atoms per molecule, 10 to 40% by weight of an iso-paraffin, 10 to 40% by weight of an n-paraffin, and 5 to 20% by weight of an aromatic. It is particularly preferred if the solvent comprises 20 to 30% by weight of an aliphatic hydrocarbon having up to 15 carbon atoms per molecule, 20 to 30% by weight of an iso-paraffin, 20 to 30% by weight of an n-paraffin, and 10 to 15% by weight of an aromatic. This includes mixtures of two or more aliphatic hydrocarbons with up to 15 carbon atoms per molecule, two or more iso-paraffins, two or more n-paraffins and/or two or more aromatics. In this embodiment, the aliphatic hydrocarbon preferably comprises n-heptane, n-octane, n-nonane and/or n-decane, and/or isomers thereof, the proportion of these aliphatic hydrocarbons in the solvent preferably being at least 51% by weight, more preferably at least 65% by weight, even more preferably at least 80% by weight, based on the proportion of all aliphatic hydrocarbons having up to 15 carbon atoms per molecule present in the solvent. In this embodiment, the proportion of cycloalkanes in the solvent is preferably below 5% by weight, more preferably below 1% by weight, even more preferably below 0.1% by weight, based on the weight of the solvent. The solvent with the aforementioned composition preferably has a boiling range of 25 to 200° C., and/or a density at 15° C. of 0.70 to 0.76g/cm³. This solvent is particularly suitable for dissolving the wax. The solvent with this composition can also be obtained in the pyrolysis of plastic waste and can be used for further use in the method according to the invention.

The ratio of pyrolysis residue to solvent can preferably be in the range of 3:1 to 1:5, more preferably in the range of 1:1 to 1:3. This allows the wax to dissolve in the solvent quickly and as completely as possible.

At least a portion of the solid material is separated from the high-solid mixture in step (c) of the method, wherein the separation can take place by means of a first separation device. Due to the at least partial separation of the solid material, a low-solid mixture is obtained. A solids concentration of the low-solid mixture obtained is preferably at most 30% by weight, more preferably at most 15% by weight, even more preferably at most 5% by weight, even more preferably at most 2% by weight, even more preferably at most 1% by weight, based on the total weight of the low-solid mixture.

The separation of the solid material can be carried out by gravimetric separation, filtration and/or centrifugal separation from the high-solid mixture. Gravimetric separation may include sedimentation and/or decantation. In particular, the separation can be carried out by filtration. The filtration may be carried out with a filter medium, which may comprise an activated carbon or a bleaching earth. As a result, any polar components (e.g. a tar or a polyphenol) dissolved in the solvent together with the wax can be well separated and adsorbed by the filter medium. During filtration, even particles with an average particle size (D50) of less than 100 μm, in particular less than 50 μm, can be easily removed (determined by laser diffractometry). The filtration is preferably carried out at elevated temperature. The filtration is preferably carried out at a temperature in the range from 80 to 350° C., more preferably from 90 to 300° C., even more preferably from 100 to 250° C., most preferably from 150 to 180° C. It is also preferred if the filtration is carried out at a pressure of 5 bar or above, preferably at 5 to 20 bar, more preferably at 10 to 20 bar. Increasing the temperature and/or reducing the pressure allows for more efficient separation of the solid material from the high-solid mixture.

In a preferred embodiment, the separation of the solid material takes place by sedimentation and subsequent filtration, the filtration preferably taking place at a pressure of 5 bar or more and at a temperature in the range from 100 to 250° C., even more preferably at a pressure of 10 bar or more and at a temperature in the range from 150 to 180° C. With these parameters, the solid material can be separated particularly well from the high-solid mixture. This separation efficiency can be further improved if the aliphatic hydrocarbon contained in the solvent is n-heptane, and in particular if the solvent is n-heptane.

The solid material preferably comprises an inorganic salt, a ceramic raw material, an asphaltene, a tar, and/or a coke. Coke is particularly preferably contained in the pyrolysis residue.

This is particularly the case if the pyrolysis residue is obtained by pyrolyzing a plastic, and subsequently a liquid fraction and/or a gaseous fraction are separated from the pyrolysis residue before the pyrolysis residue according to step (a) of the method is provided. In particular, the solid material may comprise talc, an iron oxide (e.g., ferric oxide), aluminum oxide, titanium dioxide, magnesium oxide, and/or calcium carbonate. If the provided pyrolysis residue is obtained by pyrolyzing a plastic, the solid material may comprise an additive contained in the plastic. For example, the additive may comprise a filler, a color pigment, and/or an additive. A specialist knows which additives are used depending on the respective plastic and field of application.

After it has been separated from the high-solid mixture, i.e. after step (c) of the method, the solid material can be dried, for example in an oven. As a result, the solid material can be made free-flowing and thus easier to process. The solid material is preferably dried at a temperature in the range from 50 to 250° C., more preferably from 100 to 200° C., even more preferably from 130 to 160° C. The drying time is preferably up to 120 min, in particular 5 to 60 min.

One or more components can be separated from the solid material, in particular from the dried solid material, e.g. by filtration and/or centrifugation. Separated components can then be reused, e.g. as an additive for a plastic.

Solvent separated during drying of the solid material may be reused in step (b) of the method. Before being recycled into the method, the solvent can additionally be purified, preferably by evaporation, in particular by rotary evaporation.

The separation of at least a portion of the wax in step (d) of the method may comprise evaporation or extraction under pressure. The temperature in step (d) is preferably in the range from 50 to 280° C., more preferably from 80 to 150° C., particularly preferably from 100 to 130° C. The pressure in step (d) is preferably in the range from 0.1 to 2.5 bar, preferably from 0.5 to 1 bar. As a result, the separation can take place as completely and quickly as possible.

If the boiling temperature (or an upper end of the boiling range) of the solvent is significantly below an extraction temperature (e.g. at least 10° C. below, or at least 15° C. below), the separation preferably comprises extraction under pressure, for example in a hydrocyclone. If the boiling temperature (or a lower end of the boiling range) of the solvent is above the extraction temperature, substantially equal to the extraction temperature, or slightly below (e.g., up to 10° C. below, or up to 15° C. below), the separation preferably comprises evaporation.

After separating the wax in step (d) of the method, solvent contained in the low-solid mixture can be reused in step (b). For this purpose, the solvent can be separated from the low-solid mixture, preferably by evaporation, in particular by rotary evaporation.

The wax separated in step (d) of the method preferably has a boiling temperature (or a lower end of a boiling range) of at least 280° C., more preferably at least 300° C., even more preferably at least 350° C. The wax preferably has a boiling temperature (or a boiling range) in the range from 280 to 730 ° C., even more preferably from 350 to 700° C. The wax can then be easily separated from the low-solid mixture. The boiling temperature (or the boiling range) of the wax can be determined by means of the standard ASTM D7500-15: 2019 (preferably for a wax with a boiling temperature or a boiling range of 100 to 850° C., in particular 100 to 735° C.) or by means of the standard ASTM D 2887-22:2022 (preferably for a wax with a boiling temperature or a boiling range of 55 to 538° C.)

The separated wax preferably has at least 15 carbon atoms per molecule, more preferably at least 20, in particular at least 40. The wax preferably has 20 to 80 carbon atoms per molecule, in particular 20 to 65. Such waxes can be easily reused in a refinery, as a lubricant and/or as an additive.

The separated wax preferably comprises at least 0.1% by weight of the solvent, more preferably at least 5% by weight, in particular at least 10% by weight, based on the total weight of the wax. This can reduce the setting point of the wax and thus improve its pumpability. The separated wax preferably comprises a maximum of 60% by weight of the solvent, preferably a maximum of 50% by weight, more preferably a maximum of 20% by weight, based on the total weight of the wax. Otherwise, the properties of the wax may be impaired. The separated wax preferably comprises 0.1 to 50% by weight of the solvent, more preferably 0.1 to 20% by weight, based on the total weight of the wax.

The separated wax can be supplied to a processing plant of a refinery, in particular a fluid catalytic cracking (FCC) plant, a steam cracker, a thermal gasoil unit (TGU plant), a hydrogenation plant and/or a coker. Preferably, the wax is supplied to an FCC plant and/or a steam cracker. In a steam cracker, the wax can be converted into a low molecular weight alkene (or into a mixture of two or more such alkenes), in particular into ethene and/or propene. This enables reuse in the production of plastics, in particular polyolefins.

When fed to the processing plant, the wax preferably has a temperature of 40° C. or above, preferably of 50° C. or above, more preferably 80° C. or above, in particular 100° C. or above; the temperature is preferably from 40 ° C. to 300° C., preferably from 50° C. to 280° C., even more preferably from 80 to 200° C., even more preferably from 100 to 180° C. Then a stagnation of the wax can be avoided. This can also be advantageous for energy reasons if the separation of at least a portion of the wax from the low-solid mixture already takes place at an elevated temperature, and an elevated temperature is also required in the processing plant, because the already heated wax can then be fed to the processing plant without prior cooling, and optionally with further heating. The separated wax can also be used in other technical areas, for example as a lubricant and/or additive. The separated wax can be reprocessed before further use. For example, the wax can be purified and/or separated into different carbon fractions.

Preferably, the separated wax is mixed with a hydrocarbon stream before being supplied to a processing plant of a refinery, in particular an FCC plant. The hydrocarbon stream preferably comprises a gas oil, preferably a vacuum gas oil (VGO) and/or a visbreaker gas oil. The hydrocarbon stream preferably has a boiling temperature (or a lower end of a boiling range) of 100° C. or above, more preferably 150° C. or above, even more preferably 200° C. or above. It is particularly preferred if the hydrocarbon stream has a boiling temperature (or a boiling range) in the range from 100 to 400° C., more preferably from 150 to 300° C., even more preferably from 150 to 200° C. This can result in optimal rheological properties for further processing of the mixture of the wax with the hydrocarbon stream. The boiling temperature (or the boiling range) of the hydrocarbon stream can be determined by means of the standard ASTM D7500-15: 2019 (preferably for a hydrocarbon stream with a boiling temperature or a boiling range of 100 to 850° C., in particular 100 to 735° C.) or by means of the standard ASTM D 2887-22:2022 (preferably for a hydrocarbon stream with a boiling temperature or a boiling range of 55 to 538° C.). Preferably, the hydrocarbon stream has a simi-lar number of carbon atoms per molecule as the separated wax (e.g., a number of 10 carbon atoms or more per molecule). Then the wax can be mixed well with the hydrocarbon stream. The mixture of the wax and the hydrocarbon stream preferably contains an amount of the wax of 50% by weight or less, more preferably 20% by weight or less, even more preferably 10% by weight or less, particularly preferably 5% by weight or less, in particular 2% by weight or less, based on the total weight of the mixture. The mixture particularly preferably contains an amount of the wax of 0.1 to 2% by weight, based on the total weight of the mixture. This not only enables the separated wax to be up-graded, but the mixing of the wax with the hydrocarbon stream can also ensure constant process conditions in the processing plant of the refinery, in particular the FCC plant.

The invention also relates to a device for obtaining a wax from the pyrolysis residue using the method according to the invention, comprising
an introduction device for adding the solvent to the pyrolysis residue,
a first separation device for separating at least a portion of the solid material from the high-solid mixture, and
a second separation device for separating at least a portion of the wax from the low-solid mixture,
preferably wherein a cooling unit (11) for cooling the pyrolysis residue is arranged upstream of the introduction device (14) prior to the addition of the solvent.

The introduction device for adding the solvent may comprise a metering device, such as a metering pump.

The introduction device can be connected to a container to which the pyrolysis residue can be fed, so that the dissolving of the wax in the solvent can take place in this container. The container may be heatable to allow the wax to dissolve in the solvent at an elevated temperature.

The first separation device may comprise a gravimetric separation device, a filter, a cyclone, a hydrocyclone, or a combination thereof, preferably a filter.

The second separation device may comprise an evaporator and/or a device for extraction.

The device may further comprise a dryer, such as an oven, in particular a vacuum oven, in order to dry the solid material after its separation, i.e. after step (c).

The invention further relates to a device for obtaining a wax from a plastic, in particular from a waste plastic, comprising the aforementioned device (for obtaining a wax from the pyrolysis residue) and a pyrolysis reactor for pyrolyzing the plastic in order to obtain the pyrolysis residue.

The device may further comprise a mixer, in particular an extruder, to plasticize the plastic prior to addition to the pyrolysis reactor.

The device may also comprise a feed device for adding a diluent to the plastic and thereby reducing its viscosity, wherein the feed device is connected upstream of the pyrolysis reactor.

If the device has an extruder, the feed device can be connected downstream of the extruder or arranged directly on the extruder, in particular in its compression zone or mixing zone. Alternatively, the device may comprise a mixer, in particular a static mixer, for mixing the plastic, in particular the plasticized plastic, with the diluent.

The invention relates in particular to the following embodiments:

1. A method for obtaining a wax from a pyrolysis residue, comprising the steps of
(a) providing the pyrolysis residue, wherein the pyrolysis residue comprises the wax and a solid material, preferably wherein the solid material comprises an inorganic salt, a ceramic raw material, an asphaltene, a tar and/or a coke,
(b) adding a solvent to the pyrolysis residue to obtain a high-solid mixture comprising the solid material and the solvent, wherein the wax is at least partially dissolved in the solvent,
(c) separating at least a portion of the solid material from the high-solid mixture to obtain a low-solid mixture, and
(d) separating at least a portion of the wax from the low-solid mixture.

2. The method according to embodiment 1, wherein the solvent has a boiling temperature in the range from 20 to 270° C., preferably from 20 to 250° C., more preferably from 35 to 255° C., in particular from 50 to 150° C.

3. The method according to embodiment 1 or 2, wherein the solvent comprises at least 10% by weight, preferably at least 20% by weight, more preferably at least 50% by weight, even more preferably at least 70% by weight, in particular at least 90% by weight, of an aliphatic hydrocarbon, based on the total weight of the solvent.

4. The method according to embodiment 3, wherein the aliphatic hydrocarbon is selected from the group of aliphatic hydrocarbons having up to 15 carbon atoms per molecule, preferably from 4 to 12 carbon atoms per molecule.

5. The method according to embodiment 4, wherein the aliphatic hydrocarbon is selected from n-pentane, n-hexane, cyclo-hexane, n-heptane, iso-dodecane, tetradecane, an isomer thereof, or a mixture thereof.

6. The method according to any one of embodiments 1 to 5, wherein the ratio of pyrolysis residue to solvent is in the range of 3:1 to 1:5, preferably in the range of 1:1 to 1:3.

7. The method according to any one of embodiments 1 to 6, wherein in step (b) the dissolving of the wax in the solvent takes place at a temperature of at least 80° C., preferably at least 100° C., more preferably at least 120° C., even more preferably at least 140° C., in particular at least 160° C., particularly preferably in the range from 80 to 240° C., more preferably from 100 to 160° C., in particular 100 to 140° C.

8. The method according to any one of embodiments 1 to 7, wherein in step (b) the dissolving the wax in the solvent takes place at a pressure in the range of 1 to 25 bar, preferably in the range of 5 to 16 bar.

9. The method according to any one of embodiments 1 to 8, wherein the solid material is separated in step (c) by gravimetric separation (in particular sedimentation), filtration, and/or centrifugal separation, preferably by filtration.

10. The method according to embodiment 9, wherein the solid material is separated in step (c) by filtration, preferably by sedimentation and subsequent filtration; preferably wherein the filtration is carried out at a temperature in the range from 80 to 350° C., more preferably from 90 to 300° C., even more preferably from 100 to 250° C., most preferably from 150 to 180° C.; and/or at a pressure of 5 bar or above, preferably at 5 to 20 bar, more preferably at 10 to 20 bar.

11. The method according to any one of embodiments 1 to 10, wherein the solid material is dried after step (c), preferably at a temperature of 50 to 250° C., more preferably of 100 to 200° C., in particular of 130 to 160° C., and/or for a period of up to 120 min, preferably of 5 to 60 min.

12. The method according to embodiment 11, wherein solvent separated during drying of the solid material is reused in step (b).

13. The method according to any one of embodiments 1 to 12, wherein separating at least a portion of the wax in step (d) comprises evaporation or extraction under pressure.

14. The method according to embodiment 13, wherein the temperature is in the range from 50 to 280° C., more preferably from 80 to 150° C., particularly preferably from 100 to 130° C., and/or wherein the pressure is in the range from 0.1 to 2.5 bar, more preferably from 0.5 to 1 bar.

15. The method according to any one of embodiments 1 to 14, wherein solvent contained in the low-solid mixture after step (d) is reused in step (b).

16. The method according to any one of embodiments 1 to 15, wherein the separated wax has a boiling temperature of at least 280° C., preferably at least 300° C., more preferably at least 350° C., wherein the separated wax preferably has a boiling temperature in the range of 280 to 730° C., more preferably from 350 to 700° C.

17. The method according to any one of embodiments 1 to 16, wherein the separated wax has at least 15 carbon atoms per molecule, preferably at least 20, in particular at least 40, wherein the wax preferably has 20 to 80 carbon atoms per molecule, in particular 20 to 65.

18. The method according to any one of embodiments 1 to 17, wherein the wax separated in step (d) comprises at least 0.1% by weight of the solvent, more preferably at least 5% by weight, even more preferably at least 10% by weight, and/or wherein the separated wax comprises a maximum of 60% by weight of the solvent, preferably a maximum of 50% by weight, more preferably a maximum of 20% by weight, based on the total weight of the wax.

19. The method according to embodiment 18, wherein the separated wax comprises 0.1 to 50% by weight of the solvent, more preferably 0.1 to 20% by weight, based on the total weight of the wax.

20. The method according to any one of embodiments 1 to 19, wherein the pyrolysis residue is obtained by pyrolyzing a plastic, in particular a waste plastic.

21. The method according to embodiment 20, wherein the pyrolysis residue is obtained by pyrolyzing the plastic in a pyrolysis reactor and subsequently increasing a solids concentration of the pyrolysis residue.

22. The method according to embodiment 20 or 21, wherein the pyrolysis residue comprises a liquid fraction, wherein the proportion of the liquid fraction is preferably at most 95% by weight, more preferably in the range from 30 to 95% by weight, in particular from 50 to 88% by weight, based on the total weight of the pyrolysis residue.

23. The method according to any one of embodiments 20 to 22, wherein the pyrolysis residue is cooled before step (b), preferably to a temperature of not more than 260° C., more preferably not more than 220° C., especially preferably not more than 180° C., and/or preferably to a temperature of not less than 80° C., more preferably not less than 100° C., especially preferably not less than 120° C.

24. The method according to any one of embodiments 1 to 23, wherein the separated wax is supplied to a processing plant of a refinery, in particular an FCC plant and/or a steam cracker; preferably wherein the wax, when supplied to the processing plant, has a temperature of 40° C. or above, preferably of 50° C. or above, more preferably 80° C. or above, in particular 100° C. or above; or a temperature of 40° C. to 300° C., preferably of 50° C. to 280° C., even more preferably of 80 to 200° C., even more preferably of 100 to 180° C.

25. The method according to embodiment 24, wherein the separated wax is mixed with a hydrocarbon stream before being supplied to the processing plant, in particular the FCC plant; wherein the hydrocarbon stream preferably has a boiling temperature (or a lower end of a boiling range) of 100° C. or above, more preferably of 150° C. or above, even more preferably of 200° C. or above; and/or wherein a mixture of the wax and the hydrocarbon stream preferably contains an amount of the wax of 50% by weight or below, more preferably 20% by weight or below, even more preferably 10% by weight or below, especially preferably 5% by weight or below, in particular 2% by weight or below, based on the total weight of the mixture.

26. A device for obtaining a wax from a pyrolysis residue using the method according to any one of embodiments 1 to 25, comprising
an introduction device for adding the solvent to the pyrolysis residue, and
a first separation device for separating at least a portion of the solid material from the high-solid mixture, and
a second separation device for separating at least a portion of the wax from the low-solid mixture,
preferably wherein a cooling unit (11) for cooling the pyrolysis residue is preferably arranged upstream of the introduction device (14) prior to the addition of the solvent.

27. The device according to embodiment 26, wherein the introduction device comprises a metering device, in particular a metering pump.

28. The device according to embodiment 26 or 27, wherein the first separation device comprises a gravimetric separation device, a filter, a cyclone, a hydrocyclone, or a combination thereof, preferably a filter.

29. The device according to any one of embodiments 26 to 28, wherein the second separation device comprises an evaporator and/or a device for extraction.

30. A device for obtaining a wax from a plastic, in particular from a waste plastic, comprising the device according to any of embodiments 26 to 29 and a pyrolysis reactor for pyrolyzing the plastic in order to obtain the pyrolysis residue.

31. The device according to embodiment 30, further comprising a mixer, preferably an extruder, to plasticize the plastic prior to addition to the pyrolysis reactor.

32. The device according to embodiment 30 or 31, further comprising a feed device for adding a diluent to the plastic, wherein the feed device is connected upstream of the pyrolysis reactor.

The invention is explained in more detail below with reference to descriptions of figures of some embodiments, to which the invention is not limited, however.

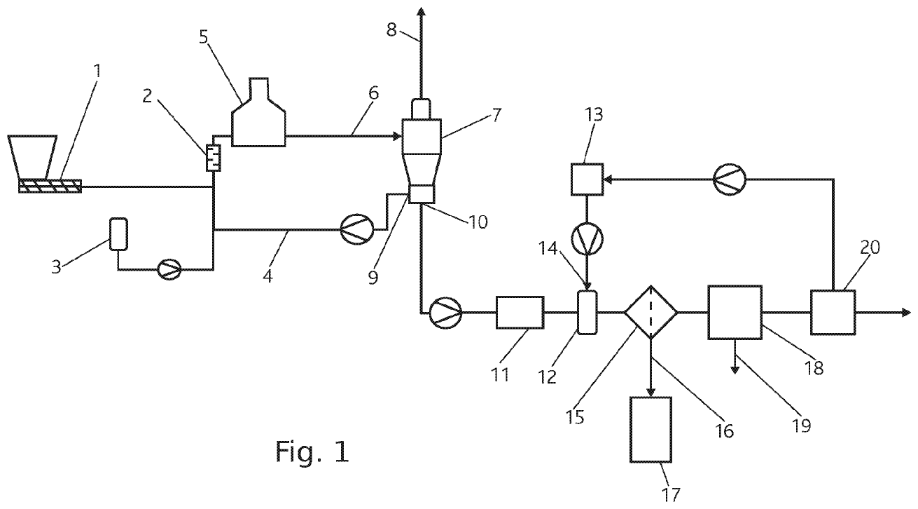

FIG. 1 shows a flow chart of a pyrolysis method wherein wax is obtained from a pyrolysis residue.

As can be seen from FIG. 1, a plastic comprising at least 50% by weight of a polyolefin is fed to an extruder 1, in which the plastic is plasticized and degassed. The plasticized plastic has a temperature of at least 120° C. and is then added to a static mixer 2. In the static mixer 2, a diluent 3 can be added to the plasticized plastic in order to reduce its viscosity. Alternatively or in addition to the diluent 3, a portion of a liquid fraction 4 separated from a pyrolysis residue may be mixed with the plasticized plastic to reduce its viscosity. The obtained mixture is then fed to a pyrolysis reactor 5, in which the plastic is pyrolyzed at a temperature of 350 to 450° C. As a result, a pyrolysis product 6 comprising a gaseous fraction, a liquid fraction, and a wax and a solid material is obtained. The pyrolysis product 6 is fed to a hydrocyclone 7 downstream of the pyrolysis reactor 5. First, the gaseous fraction is at least partially separated in the hydrocyclone 7. The separated portion of the gaseous fraction 8 can then be further separated into a light oil (e.g. with a boiling range of 35 to 225° C.) and a heavy oil (e.g. with a boiling range of 225 to 410° C.) (not shown). Furthermore, the liquid fraction is at least partially separated in the hydrocyclone 7. The separated portion of the liquid fraction 4 can be discharged via an outlet 9 of the hydrocyclone 7 and reused in the method for viscosity reduction of the plastic, as described above. At least a portion of the pyrolysis residue, which at least partially comprises the wax and the solid material, is discharged via an outlet 10 arranged in the bottom of the hydrocyclone 7.

As can also be seen in FIG. 1, the pyrolysis residue discharged via the outlet 10 is fed to a cooling unit 11 for cooling to a temperature in the range from 80 to 240° C. Subsequently, a solvent 13 comprising at least 20% by weight of an aliphatic hydrocarbon is added to the pyrolysis residue in a container 12 by means of an introduction device 14 in order to obtain a high-solid mixture. The aliphatic hydrocarbon is selected from the group of aliphatic hydrocarbons having 4 to 12 carbon atoms per molecule. The obtained high-solid mixture has a ratio of pyrolysis residue to solvent in the range from 1:1 to 1:3. The container 12 is heated so that the dissolving can take place at a temperature in the range of 80 to 240° C. In a first separation device 15 comprising a filter, the solid material is at least partially separated from the high-solid mixture to obtain a low-solid mixture. The separated solid material 16 is then dried in an oven 17 at a temperature in the range from 100 to 200° C. Subsequently, individual components can be separated from the solid material and reused (not shown). The low-solid mixture is fed to a second separation device 18, which has an evaporator, in order to separate the wax at least partially by means of evaporation. Evaporation takes place at a temperature in the range of 80 to 150° C. and a pressure of 0.5 to 1 bar. The separated wax 19 can then be put to further use. At least a portion of the solvent is subsequently separated from the low-solid mixture in an evaporator 20 and reused in step (b) of the method by returning it to the container 12 via the introduction device 14.

1. A method for obtaining a wax from a pyrolysis residue, comprising the steps of
(a) providing the pyrolysis residue, wherein the pyrolysis residue comprises the wax and a solid material, and wherein the solid material comprises an inorganic salt, a ceramic raw material, an asphaltene, a tar and/or a coke,
(b) adding a solvent to the pyrolysis residue to obtain a high-solid mixture comprising the solid material and the solvent, wherein the wax is at least partially dissolved in the solvent,
(c) separating at least a portion of the solid material from the high-solid mixture to obtain a low-solid mixture, and
(d) separating at least a portion of the wax from the low-solid mixture.

2. The method according to claim 1, wherein the solvent comprises at least 20% by weight of an aliphatic hydrocarbon, based on the total weight of the solvent.

3. The method according to claim 2, wherein the aliphatic hydrocarbon is selected from the group of aliphatic hydrocarbons having up to 15 carbon atoms per molecule, preferably from 4 to 12 carbon atoms per molecule.

4. The method according to claim 3, wherein the aliphatic hydrocarbon is selected from n-pentane, n-hexane, n-heptane, iso-dodecane, tetradecane, or a mixture thereof.

5. The method according to claim 1, wherein in step (b), the dissolving of the wax in the solvent takes place at a temperature of at least 80° C.

6. The method according to claim 1, wherein the solid material is separated in step (c) by sedimentation and subsequent filtration.

7. The method according to claim 6, wherein the filtration is carried out at a temperature in the range of 100 to 250° C. and a pressure of 5 bar or above.

8. The method according to claim 1, wherein the solid material is dried after step (c).

9. The method according to claim 1, wherein separating at least a portion of the wax in step (d) comprises evaporation or extraction under pressure.

10. The method according to claim 1, wherein solvent contained in the low-solid mixture after step (d) is reused in step (b).

11. The method according to claim 1, wherein the separated wax has a boiling temperature of at least 280° C.

12. The method according to claim 1, wherein the wax separated in step (d) comprises at least 0.1% by weight of the solvent.

13. The method according to claim 1, wherein the pyrolysis residue is obtained by pyrolyzing a plastic, in particular a waste plastic.

14. A device for obtaining a wax from a pyrolysis residue using the method according to claim 1, comprising
an introduction device for adding the solvent to the pyrolysis residue, and
a first separation device for separating at least a portion of the solid material from the high-solid mixture, and
a second separation device for separating at least a portion of the wax from the low-solid mixture,
wherein a cooling unit for cooling the pyrolysis residue is arranged upstream of the introduction device prior to the addition of the solvent.

15. A device for obtaining a wax from a plastic, in particular from a waste plastic, comprising the device according to claim 14 and a pyrolysis reactor for pyrolyzing the plastic in order to obtain the pyrolysis residue.
